(19) United States
(12) Patent Application Publication
(10) Pub. No.: US 2008/0020369 A1
(43) Pub. Date: Jan. 24, 2008

(54) NEUROTOXICITY METHODS PROCESS

(76) Inventors: Kristen E. Drake, (US); Sandra M. Hooper, (US); Tracy Cameron, (US); Bruce Horowitz, (US)

Correspondence Address: ADVANCED NEUROMODULATION SYSTEMS, INC., 6901 PRESTON ROAD, PLANO, TX, 75024, US

(21) Appl. No.: 11/552,028

(22) Filed: Oct. 23, 2006

Related U.S. Application Data
(60) Provisional application No. 60/730,809, filed on Oct. 27, 2005.

(51) Int. Cl. C12Q 1/00; C12N 5/08

(52) U.S. Cl. 435/4; 435/368

(57) ABSTRACT

A method for testing the cytotoxicity of an implant comprising: providing neuronal cells in-vitro; providing one or more compounds of the implantable medical device; adding the one or more compounds from the implantable medical device to the in-vitro neuronal cells; and determining a measure of the cytotoxicity of the one or more compounds based on an assessment of the neuronal cells.

### CROSS-REFERENCE TO RELATED APPLICATIONS

The present application claims the benefit of U. S. Provisional Application Ser. No. 60/730,809, entitled “NEUROTOXICITY METHODS PROCESS” filed Oct. 27, 2005.

### TECHNICAL FIELD

The present invention relates generally to cytotoxicity testing and more particularly to cytotoxicity testing using neuronal cell lines.

### BACKGROUND

Implants such as medical devices, prostheses, stents, leads, etc. are increasingly prevalent tools for treating patients suffering from, for example, disease, chronic ailments, pain, and other similar conditions. While implants are an important treatment tool, patient safety must remain a foremost consideration. Accordingly, a variety of testing procedures for testing implants have been developed to ensure that risk to patients using implants is minimized.

Many implant testing procedures test for biocompatibility to ensure that an implant is comprised of materials that do not cause undesired reactions in patients. Certain implants may be subject to tests required by government or regulatory entities charged with oversight and approval, such as the U.S. Food and Drug Administration. Other implant testing procedures may be voluntarily performed by manufacturers during the development process.

Many required and voluntary testing procedures are very expensive to implement, particularly those that use animals for testing. For example, even a very basic test protocol using primates can cost hundreds of thousands of dollars. Because testing costs decrease when using smaller and/or less complex species such as mice or rats, many conventional implant tests use non-primate testing to approximate patient responses. However, the use of animal species not closely related to the patient species increases the risk that the results of a test protocol will not approximate a patient's response.

Conventional animal testing protocols are also objectionable for time considerations. Many animal protocols take many weeks or months to perform, and often entail objectionable procedures that result in pain and/or the death of animal test subjects. Also, data from animal testing protocols often does not provide a direct correlation to results that will be seen in a human application. Accordingly, the use of testing protocols that eliminate or substitute for animal protocols is desirable.

### SUMMARY

Representative embodiments are to neuronal cytotoxicity testing for implantable medical devices. A neuronal cytotoxicity test may be used to establish whether an implant, such as a stimulation lead, is likely to have toxic effects on a patient's tissue when implanted.

Neuronal cells are first cultured in-vitro according to a predetermined culture protocol. An implant to be tested or a sample from an implant to be tested is incubated with an extraction medium. In many embodiments of the present invention, the extraction medium is a physiologically-compatible liquid selected to prevent any effects on the in-vitro neuronal cells when added to the cells. The incubation period, temperature, and environment may be varied in certain embodiments of the present invention. Other experimental parameters may be varied or selected to best approximate the environment in which the implant being tested will be used. After incubation, the extraction medium is then added to the neuronal cells in-vitro and incubated with the cells for a predetermined period of time.

Following the incubation of the neuronal cells and the extraction medium, the neuronal cells are then assessed. Both quantitative and qualitative assessments may be performed on the neuronal cells. The assessments are then used to determine the cytotoxicity of the implant. In preferred embodiments of the present invention, the implant tested are implantable medical devices for neurostimulation.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 is a flow diagram of a method according to one embodiment of the present invention; and

FIG. 2 is a flow diagram of an extraction procedure comprising an embodiment of the present invention.

### DETAILED DESCRIPTION

Biocompatibility tests are performed on implants to determine whether the implant poses a risk when used by a patient. Cytotoxicity test protocols are often performed as part of broader biocompatibility tests. Generally, cytotoxicity test protocols test for deleterious effects at a cellular level. For example cytotoxicity test protocols may test for whether an implant comprises materials or has properties that induce damage or undesirable effects to a user's tissue. Examples of such testing protocols are outlined in ISO-10993 “Biological Evaluation of Medical Devices Part 1: Evaluation and Testing,” herein incorporated by reference.

Generally, embodiments of the present invention comprise testing methods for testing an implant for cytotoxicity. In these embodiments, neuronal cells are grown in a suitable growth medium. A compound or compounds removed from an implant being tested are then added to the neuronal cells or the growth medium surrounding the cells. The neuronal cells are then assessed using one or more quantitative or qualitative assessment methods to determine the cytotoxicity of the compound(s) added to the cells.

Certain embodiments of the present invention are used at various development stages such as basic material testing, final finished goods testing, or other testing or evaluation stages. Also, certain embodiments may comply with many regulatory agency requirements for basic materials testing. Compared to conventional animal studies typically required by regulatory agencies for basic material testing, embodiments of the present invention may provide cost and/or time savings. Thus, embodiments of the present invention can lower overall research and development costs and time requirements.

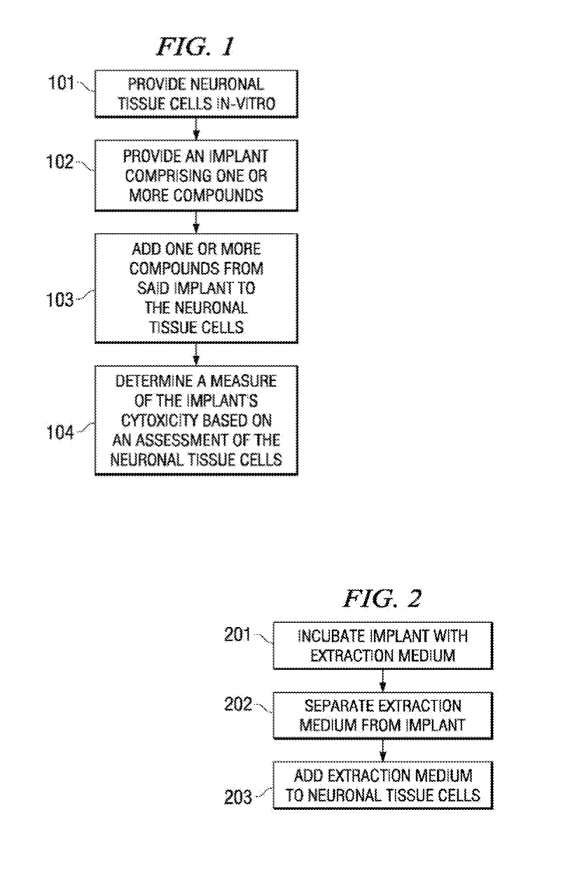

Referring now to FIG. 1, process of performing neurotoxicity testing according to representative embodiments of the present invention is shown. In step 101, neuronal cells are provided in-vitro. In preferred embodiments of the present invention, the neuronal cells are human neuronal cells. However, neuronal cells from other species may be provided in-vitro in other embodiments. Neuronal cells from various nervous system tissues can be provided, such as, for example, glial tissue, cortical tissue, and cerebellar tissue. Neuronal cells may be provided in-vitro from cultures of widely available neuronal cells lines such as those available through the American Type Culture Collection (Manassas, Va., hereinafter “ATCC®”). Cell lines usable with embodiments of the invention are, for example, ATCC®# HTB-186, ATCC®# CRL-2366, and ATCC®# CRL-10442.

In step 102, an implant for testing is provided. The implant could be a medical device, prosthesis, stent, lead, pacemaker, neurostimulator, etc. The implant testing occurs by isolating or identifying one or more compounds contained in the implant. For example, a leachate can be produced from the implant for the provisional to neural tissue. Alternatively, an implant may be provided in its entirety or one or several components therefrom may be provided.

In step 103, one or more compounds of the implant for testing are added to the neuronal cells provided in step 101. In a preferred embodiment, the one or more compounds are extracted from the implant for testing. In other embodiments of the present invention, one or more compounds comprising the implant for testing are identified via compound identification methods such as, for example, gas or liquid chromatography, spectrography, mass spectrometry, chemical testing, etc. Identified compounds are then added to the neuronal cells provided in step 101.

In step 104, a measure of the cytotoxicity of the implant for testing is determined based on an assessment of the neuronal cells. Such an assessment can be made immediately after adding one or more compounds to the cells in step 103 in certain embodiments. In other embodiments of the present invention, an assessment is made after a predetermined time period has passed following the addition of the one or more compounds in step 103. The predetermined time period may be selected in relation to the growth rate of the neuronal cells in certain embodiments.

Multiple assessments may be made at the same or different times following the addition of the one or more compounds. Both qualitative and quantitative assessments of the neuronal cells are made in certain embodiments of the present invention. Qualitative assessments may comprise, for example, grading of neuronal cell morphology, conformation, macrostructure, morbidity/mortality, etc. Qualitative assessments may provide results in the form of grading scales or systems in certain embodiments.

Quantitative assessments may comprise cell counting, uptake assays, absorbance measurements, etc. In a preferred embodiment, quantitative assessments comprise cell proliferation assays. Cell proliferation assays directly or indirectly measure the rate of cell growth. Direct methods may comprise, for example, observation using a microscope or cell counting methods such as flow cytometry. Indirect measurement assays may assess cell metabolic activity by measuring incorporation of various compounds. The compounds, such as thymidine, are often labeled with radioactive or chromogenic compounds. In a preferred embodiment of the present invention, a cell proliferation assay uses MTT tetrazolium dye. Such a cell proliferation assay is the CellTiter 96® AQᵤₑₒᵤₛ One Solution Cell Proliferation Assay manufactured by Promega Corporation (Madison, Wis.), the instructions for which are herein incorporated by reference.

Generally, qualitative and quantitative assessments can be correlated to cytotoxic effects of the one or more compounds added to the neuronal cells in step 103. For example, a reduction in the number of viable neuronal cells after the addition of the one or more compounds, as measured by a cell proliferation assay, would indicate cytotoxic effects. Also, changes in cell morphology could indicate undesired cytotoxic effects.

FIG. 2 shows a process of generating an extraction method for neurotoxicity testing according to representative embodiments of the present invention. Various extraction media in solid, liquid, and gaseous phases are used with embodiments of the present invention to produce a leachate comprising one or more compounds present in an implant for testing. In step 201, an implant for testing is incubated with extraction medium to form a leachate. In certain embodiments, more than one extraction medium is used. For example, implants are incubated in parallel in a saline medium and in a solvent medium to generate two leachates.

The leachate produced as a result of incubation of an implant with extraction medium may comprise a relatively low quantitative amount of the one or more compounds, but the amount of the compounds could potentially have a significant neurotoxic effect. In a preferred embodiment, a physiologically-compatible medium is used such as, for example, saline or a cell-culture media such as Modified Eagle's Medium supplemented with fetal bovine serum. Extraction media may be selected to approximate the environment in which the implant will be used. In general, a sterile environment is maintained during incubation in step 201. The duration and temperature at which incubation is performed may vary, and are based on the chemical stability of that material being tested. In a preferred embodiment, the temperature selected is chosen to approximate the normal temperature range of a human, or around 37 degrees Celsius. Also in a preferred embodiment, the incubation period is at least 24 hours. Temperature variations can be made during incubation to better approximate the application environment of the implant for testing.

In step 202, the extraction medium is separated from the implant with which it was incubated. Sterile technique is maintained in step 202 in embodiments of the present invention.

In step 203, extraction medium separated in step 202 is added to neuronal cells cultured in-vitro. Various dilutions of the extraction medium can be added to the neuronal cells in certain embodiments of the present invention. Also, additional compounds may be added to the extraction medium before addition to the neuronal cells. Once the extraction medium has been added to the neuronal cells, a measure of the implant's cytotoxicity can be determined as described above in step 104 of FIG. 1.

### Cytotoxicity Testing Protocols

Described below are three neuronal cell cytotoxicity testing protocols according to embodiments of the present invention. The protocols were conducted in compliance with Good Laboratory Practice (GLP) regulations known to those of skill in the art.

### General

An implant for testing is provided in all three protocols. In one embodiment, Advanced Neuromodulation Systems (Plano, Tex., hereinafter “ANS”) deep brain stimulation leads of various lengths and electrode spacings for neurostimulation are provided as ethylene oxide-sterilized finished products. Other implantable devices and components such as, for example, infusion catheters, etc. can be used in other embodiments. The negative control used for all three cell lines was USP Negative Bioreaction Reference Standard High Density Polyethylene (HDPE) in sheet form. The positive control used for all three cell lines was Tygon® F-4040-A in slab form. In the three protocols, the cell proliferation assay used for qualitative assessment is the Cell Titer 96® Aqueous One Solution Cell Proliferation Assay.

In these three cytotoxicity protocols, the implant for testing is a deep brain stimulation lead from final finished product stock with a removed stylet. The leads were intact and were not cut or scored. One or more compounds were extracted from the leads using extraction medium incubated with the leads as described above in FIG. 2. Leachate from the extraction was diluted to concentrations of 1:2, 1:4, 1:8, 1:16 and 1:32 that were applied directly in 2 mL aliquots to neuronal cells prepared in-vitro.

Extractions were completed in 25% control concentration, serial dilution saline “neat,” and serial dilution media extraction with serum. Negative controls were prepared by incubating USP Negative Bioreaction Reference Standard HDPE using undiluted saline or media with serum. Positive controls were prepared by incubating Tygon® F-4040-A using undiluted saline or media with serum.

Qualitative assessment results are scaled on a 5-point linear scale. Zero (0) is interpreted as “non-cytotoxic” with the cells condition being described as discrete with no lysis. Plus/minus (±) is interpreted as “minimally cytotoxic” with ≦20% cells rounded or lysed. One (1) is interpreted as “mildly cytotoxic” with ≦50% cells rounded or lysed. Two (2) is interpreted as “moderately cytotoxic” with ≦70% cells rounded or lysed. Three (3) is interpreted as “severely cytotoxic” with nearly complete destruction of the cell layers.

Quantitative assessment of test and control cultures were made using the Cell Titer 96® Aqueous One Solution Cell Proliferation Assay protocol from Promega Corporation. Upon completion of initial incubation according to the Cell Proliferation Assay, dye solution was added and the wells were again incubated at 37° C. for 4 hours, solubilized, and then incubated for approximately 48 hours for ATCC® # HTB-186 and ATCC # CRL-2366 cell lines and approximately 120 hours for the ATCC® # CRL-10442 cell line. Wells were recorded for absorbance at 490 nm using a 96-well plate reader.

### ATCC® # HTB-186 Cytotoxicity Protocol

ATCC® # HTB-186 cells were prepared according to ATCC® recommendations and in a manner to promote cell longevity. Cells were prepared in triplicate wells of sub-cultivation ratios between 1:4 and 1:6 using ATCC® Eagle's Minimum Essential Medium supplemented with 10% fetal bovine serum, rinsed with Trypsin-EDTA solution at 37° C. and renewed every 2-3 days. Test dilutions and controls were prepared by adding extraction medium from incubations with control articles or an implant for testing. The test plates were incubated for about 48 hours at 37° C./5% CO₂. Test plate wells were scored at least once following incubation using qualitative and quantitative tests described above in the text accompanying FIG. 1.

### Results—Qualitative Assessment

All wells were interpreted at 24 and 48 hours following the addition of saline or media.

### Results—Quantitative Assessment

The Cell Proliferation Assay was performed on well samples taken at 24, 48 and 72 hours following the addition of saline or media, and the absorbance measurement correlated to a measure of cell viability.

### ATCC® # CRL-2366 Cytotoxicity Protocol

ATCC® # CRL-2366 cells were prepared according to ATCC® recommendations and NPL SOP# 15B-01-modified. Cells were prepared in triplicate wells of sub-cultivation ratios between 1:4 and 1:6 using ATCC® Eagle's Minimum Essential Medium supplemented with 10% fetal bovine serum, rinsed with Trypsin-EDTA solution at 37° C. and renewed every 2-3 days. Test dilutions and controls were prepared by adding extraction medium from incubations with control articles or an implant for testing. The test plates were incubated for about 48 hours at 37° C./5% CO₂. Test plate wells were scored at least once following incubation using qualitative and quantitative tests described above in the text accompanying FIG. 1.

### Results—Qualitative Assessment

All wells were interpreted at 24 and 48 hours.

### Results—Quantitative Assessment

The Cell Proliferation Assay was performed on well samples taken at 24, 48 and 72 hours following the addition of saline or media, and the absorbance measurement correlated to a measure of cell viability.

### ATCC® # CRL-10442 Cytotoxicity Protocol

ATCC® # CRL-10442 cells were prepared according to ATCC® recommendations and NPL SOP # 15B-01-modified. Cells were prepared in triplicate wells of sub-cultivation ratios between 1:2 and 1:3 using ATCC® Eagle's Minimum Essential Medium supplemented with 10% fetal bovine serum, rinsed with Trypsin-EDTA solution at 37° C., centrifuged for 5-10 minutes at 125 g, and renewed every 2-3 days. Test dilutions and controls were prepared by adding extraction medium from incubations with control articles or an implant for testing. The test plates were incubated for about 120 hours at 37° C./5% CO₂. Test plate wells were scored at least once following incubation using qualitative and quantitative tests described above in the text accompanying FIG. 1.

### Results—Qualitative Assessment

All wells were interpreted at 120 hours.

### Results-Quantitative Assessment

The Cell Proliferation Assay was performed on well samples taken at 120 hours following the addition of saline or media, and the absorbance measurement correlated to a measure of cell viability.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method for testing the cytotoxicity of an implantable medical device comprising:
providing neuronal cells in-vitro;
providing one or more compounds of the implantable medical device;
adding the one or more compounds from the implantable medical device to the in-vitro neuronal cells; and
determining a measure of the cytotoxicity of the one or more compounds based on an assessment of the neuronal cells.

2. The method of claim 1 wherein the neuronal cells are mammalian neuronal cells.

3. The method of claim 1 wherein the neuronal cells are derived from human neuronal cells.

4. The method of claim 1 wherein the neuronal cells are selected from the group consisting of:
cortical, glial, and cerebellar tissue cells.

5. The method of claim 1 wherein the assessment comprises cell proliferation testing.

6. The method of claim 5 wherein the cell proliferation testing comprises measuring a reduction of a tetrazolium component by the in-vitro neuronal cells.

7. The method of claim 1 further comprising:
determining whether additional cytotoxicity testing is required based on the determining a measure of the cytotoxicity.

8. The method of claim 1 wherein the adding comprises adding one or more extracted compounds, the one or more compounds extracted by incubating the implantable medical device with an extraction solvent.

9. The method of claim 8 wherein the extraction solvent is selected from the group consisting of:
saline solution, a media containing fetal bovine serum, polar media, non-polar media, and alcohol.

10. The method of claim 9 wherein the one or more extracted compounds are added at a plurality of concentrations.

11. The method of claim 1 wherein the providing neuronal cells comprises providing neuronal cells that are grown to about 70% confluence.

12. The method of claim 1 wherein the implantable medical device is a device for implantation adjacent to neural tissue of a patient.

13. The method of claim 1 wherein the implantable medical device is a medical lead for stimulation of tissue.

14. The method of claim 1 wherein the implantable medical device is a catheter for brain infusion.

15. The method of claim 1 wherein the assessment comprises assessing the light absorbance of the neuronal cells.

16. The method of claim 1 wherein the assessment comprises quantitative and qualitative assessments.

17. The method of claim 1 further comprising:
incubating the neuronal cells at a predetermined temperature with the one or more compounds.

18. The method of claim 17 wherein the time for incubation of the neuronal cells is selected according to the growth rate of the neuronal cells.

19. The method of claim 1 wherein the providing comprises:
selecting a neuronal cell type according to the tissue present at the implant location of the implantable medical device.
